(19) United States
(12) Patent Application Publication
(10) Pub. No.: US 2021/0121593 A1
(43) Pub. Date: Apr. 29, 2021

(54) GREEN BIN ODOR REDUCTION COMPOSITION AND USE THEREOF

(71) Applicant: Build A Better Earth Movement Ltd., Victoria (CA)

(72) Inventors: Taylor McCarten, Victoria (CA); Harmen Zijlstra, Victoria (CA)

(73) Assignee: Build A Better Earth Movement Ltd., Victoria (CA)

(21) Appl. No.: 16/857,044

(22) Filed: Apr. 23, 2020

(30) Foreign Application Priority Data
Oct. 28, 2019 (CA) 3060465

(51) Int. Cl. A61L 9/013; A61L 9/012

(52) U.S. Cl. CPC A61L 9/013; A61L 9/012

(57) ABSTRACT

A dry composition for controlling food scrap odor is provided, the dry composition comprising at least one sawdust, at least one zeolite, at least one diatomaceous earth and at least one essential oil.

### CROSS-REFERENCE TO RELATED APPLICATIONS

This application claims the benefit of priority to Canadian Patent Application No. 3,060,465, filed Oct. 28, 2019, the contents of which are incorporated herein by reference.

### FIELD

The present technology relates to reducing odor from food scraps and other organic matter stored in green bins. More specifically, it is a heat stable, light stable dry composition that can be added to a green bin to absorb odor.

### BACKGROUND

In many areas, homeowners are encouraged to collect food scraps and other organic matter in so called “green bins”. The green bin contents are either collected by a waste collection company and then processed at a central composting facility or are composted at the homeowner's property. The food scraps and other organic matter in the green bins produce malodors.

A product called Green Bin Deodorizer and Cleaner was developed to be used in green bins to reduce odors associated with food scraps. It is a liquid that is added to the green bin. It does not reduce the odor, and simply masks it.

Another product is Natura Bio Green Deodorizer. It contains soft water, bacterial spore concentrate, alcohol ethoxylate, citral essential oil, tea tree oil, lemon essential oil and a mixture containing 5-chloro-2-methyl-2h-isothiazolin-3-one and 2-methyl-2h-isothiazol-3-one (anti-microbial). This is a liquid that has to be stored at room temperature and out of sunlight. The addition of a liquid to the green bin is not preferred. Further, the addition of antimicrobials could later inhibit microbial action during composting. Other approaches include trapping the odors in airtight bins.

In the related art of composting, as opposed to simply the storage of food waste prior to composting, United States Patent Application 20150045215 discloses that unit dose articles containing activated carbon are a convenient and economical way to control malodor resulting from in-home composting.

In terms of improving the quality of compost, United States Patent Application 20080116141 discloses that a naturally occurring zeolite is coated with a special nanophase Fe oxide, thereby forming a nanophase Fe oxide coated zeolite. The composition thus formed can be used for a variety of horticultural and floricultural end applications. When the nanophase Fe oxide coated zeolite is dosed with plant nutrients and is then mixed with a suitable potting compound, the result is a Complete Plant Growth Medium (CPGM). Blending 5% to 25% CPGM by weight with sand, peat, compost, vegetable and flower beds, and native and manufactured soils, provides a number of beneficial properties. Also, CPGM alone can be used for growing indoor plants to avoid plant pests and compost odor.

In terms of utilization of compost, U.S. Pat. No. 5,574,093 discloses a composition comprising a non-ionic surfactant, an anionic surfactant, a carboxylic acid, a volatile oil, an amine, a nitrogen source, and water. This composition is useful in treating composting material used for cultivating edible fungi such as mushrooms to significantly reduce the malodorous gases normally associated therewith. Mushrooms and other edible fungi grown on such treated compost material exhibit enhanced nutritional qualities, including lower sodium levels and increased protein levels compared to fungi grown on conventionally prepared compost.

Zeolites are known to reduce odors. EP 2 258 408 A1 discloses an absorbent article comprising an odour control system, wherein the odour control composition comprises two classes of odour control material, wherein a first class of odour control material such as silica gel, aldehydes or mesoporous zeolites, reduces odour by acting on malodours or a malodorous substance in the absorbent article and a second class of odour control material reduces odour by blocking the user's nose receptors due to the volatile nature of the materials selected, e.g. menthol.

What is needed is a heat and light stable composition for controlling odors from food scraps and other organic matter in household containers, more specifically green bins. It would be preferable if the composition was a dry composition. It would be preferable if the composition controlled and masked odors. It would be preferable if it didn't later interfere with composting, in other words, if it had little or no anti-microbials. It would be further preferable if the composition, once composted, enhanced the compost.

### SUMMARY

The present technology is a heat and light stable composition for controlling odors from food scraps and other organic matter in household containers, more specifically green bins. The composition is a dry composition. The composition both controls and masks odors. It does not later interfere with composting as it does not contain any anti-microbials. The composition enhances the compost from the food scraps and other organic matter.

In one embodiment, a dry composition for controlling food scrap odor is provided, the dry composition comprising at least one sawdust, at least one zeolite, at least one diatomaceous earth and at least one essential oil.

In the dry composition, the sawdust may be a softwood sawdust.

In the dry composition, the sawdust may be one or more of Tsuga, Picea, Pinus, Abies and Pseudotsuga sawdust.

In the dry composition, the sawdust may be Pseudotsuga sawdust.

The dry composition may have an average moisture content of less than about 12%.

In the dry composition, the essential oil may be lavender oil.

The dry composition may consist of about 15 to about 25 percent w/w zeolite, about 25 to about 40 percent w/w diatomaceous earth, about 5 to about 10 percent w/w sodium bicarbonate, about 1 percent w/w essential oil and the remainder being the sawdust.

The dry composition may consist of about 25 to about 40 percent w/w sawdust, about 25 to about 40 percent w/w diatomaceous earth, about 5 to about 10 percent w/w sodium bicarbonate, about 1 percent w/w essential oil and the remainder being zeolite.

The dry composition may consist of about 25 to about 40 percent w/w sawdust, about 5 to about 10 percent w/w/w sodium bicarbonate, about 1 percent w/w essential oil and the remainder being diatomaceous earth.

The dry composition may comprise about 36 percent w/w sawdust, about 18 percent w/w zeolite, about 36 percent w/w diatomaceous earth, about 9 percent w/w sodium bicarbonate and about 1 percent w/w essential oil.

In another embodiment, a method of reducing and masking odor from compostable matter in a household container is provided, the method comprising: adding a composition consisting of at least one sawdust, at least one zeolite, at least one diatomaceous earth and at least one essential oil to the household container; and adding compostable matter to the container.

In the method, the ratio of compostable matter to composition may be about 40 to about 1.

In another embodiment, a method of reducing and masking odor from compostable matter in a household container is provided, the method comprising: adding a first amount of a composition comprising about 36 percent w/w sawdust, about 18 percent w/w zeolite, about 36 percent w/w diatomaceous earth, about 9 percent w/w sodium bicarbonate and about 1 percent w/w essential oil to the household container; adding compostable matter to the household container; and adding a second amount of the composition on top of the compostable matter.

The method may further comprise adding a second amount of compostable matter to the household container; and adding a third amount of the composition on top of the second amount of compostable matter.

The method may further comprise sequentially adding compostable matter to the household container and sequentially adding the composition on top of the compostable matter.

In the method, the ratio of compostable matter to composition may be about 40 to about 1.

In the methods, the compostable matter may be food scraps.

### FIGURES

FIG. 1A is a schematic of a household container in which the composition of the present technology has been added in preparation for adding compostable matter;

FIG. 1B is a schematic of the household container of FIG. 1A following addition of compostable matter; and

FIG. 1C is a schematic of the household container in which the composition has been added on top of the compostable matter.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Zeolite—in the context of the present technology, zeolite has the formula of (Na₂, K₂) Al₂O₃ 10SiO₂ 8H₂O with Si/Al ratios ranging from 4 to 5. It may be natural or synthetic. Without being bound to theory, the pores and channels in the zeolite structures provide rapid diffusion of ions in solution. The size of the pore depends on the structure of zeolites. Zeolites possess high surface areas, reactive pore sites, high cation exchange capacities and cation selectivity. They are good adsorbents. The preferred zeolite of the present technology is mined at Kelowna, BC, Canada.

Essential oil—in the context of the present technology, a plant essential oil or derivative thereof may be extracted from a natural source or synthetically made and include racemic mixtures, enantiomers, diastereomers, hydrates, salts, solvates and metabolites, etc. Such oils generally contain as a major constituent an acyclic monoterpene alcohol or aldehyde, a benzenoid aromatic compound containing at least one oxygenated substituent or side chain, or a mono carbocyclic terpene generally having a six membered ring bearing one or more oxygenated substituents. Examples of suitable plant essential oils disclosed herein include, without limitation, .alpha.- or .beta.-pinene; .alpha.-campholenic aldehyde; .alpha.-citronellol; .alpha.-iso-amyl-cinnamic (e.g., amyl cinnamic aldehyde); .alpha.-pinene oxide; .alpha.-cinnamic terpinene; .alpha.-terpineol (e.g., 1-methyl-4-isopropyl-1-cyclohexen-8-ol); A-terpinene; achillea; aldehyde C16 (pure); .alpha.-phellandrene; amyl cinnamic aldehyde; amyl salicylate; anethole; anise; aniseed; anisic aldehyde; basil; bay; benzyl acetate; benzyl alcohol; bergamot (e.g., Monardia fistulosa, Monarda didyma, Citrus bergamia, Monarda punctata); bitter orange peel; black pepper; borneol; calamus; camphor; cananga oil (e.g., java); cardamom; carnation (e.g., Dianthus caryophyllus); carvacrol; carveol; cassia; castor; cedar (e.g., hinoki); cedarwood; chamomile; cineole; cinnamaldehyde; cinnamic alcohol; cinnamon; cis-pinane; citral (e.g., 3,7-dimethyl-2,6-octadienal); citronella; citronellal; citronellol dextro (e.g., 3-7-dimethyl-6-octen-1-ol); citronellol; citronellyl acetate; citronellyl nitrile; citrus unshiu; clary sage; clove (e.g., Eugenia caryophyllus); clove bud; coriander; corn; cotton seed; d-dihydrocarvone; decyl aldehyde; diethyl phthalate; dihydroanethole; dihydrocarveol; dihydrolinalool; dihydromyrcene; dihydromyrcenol; dihydromyrcenyl acetate; dihydroterpineol; dimethyl salicylate; dimethyloctanal; dimethyloctanol; dimethyloctanyl acetate; diphenyl oxide; dipropylene glycol; d-limonene; d-pulegone; estragole; ethyl vanillin (e.g., 3-ethoxy-4-hydrobenzaldehyde); eucalyptol (e.g., cineole); Eucalyptus citriodora; Eucalyptus globulus; Eucalyptus; eugenol (e.g., 2-methoxy-4-allyl phenol); evening primrose; fenchol; fennel; Ferniol™; fish; florazon (e.g., 4-ethyl-.alpha., .alpha.-dimethyl-benzenepropanal); galaxolide; geraniol (e.g., 2-trans-3,7-dimethyl-2,6-octadien-8-ol); geraniol; geranium; geranyl acetate; geranyl nitrile; ginger; grapefruit; guaiacol; guaiacwood; gurjun balsam; heliotropin; herbanate (e.g., 3-(1-methyl-ethyl) bicyclo(2,2,1) hept-5-ene-2-carboxylic acid ethyl ester); hiba; hydroxycitronellal; i-carvone; i-methyl acetate; ionone; isobutyl quinoleine (e.g., 6-secondary butyl quinoline); isobornyl acetate; isobornyl methylether; isoeugenol; isolongifolene; jasmine; jojoba; juniper berry; lavender; lavandin; lemon grass; lemon; lime; limonene; linallol oxide; linallol; linalool; linalyl acetate; linseed; Litsea cubeba; l-methyl acetate; longifolene; mandarin; mentha; menthane hydroperoxide; menthol crystals; menthol laevo (e.g., 5-methyl-2-isopropyl cyclohexanol); menthol; menthone laevo (e.g., 4-isopropyl-1-methyl cyclohexan-3-one); methyl anthranilate; methyl cedryl ketone; methyl chavicol; methyl hexyl ether; methyl ionone; mineral; mint; musk ambrette; musk ketone; musk xylol; mustard (also known as allylisothio-cyanate); myrcene; nerol; neryl acetate; nonyl aldehyde; nutmeg (e.g., Myristica fragrans); orange (e.g., Citrus aurantium dulcis); orris (e.g., Iris florentina) root; para-cymene; para-hydroxy phenyl butanone crystals (e.g., 4-(4-hydroxyphenyl)-2-butanone); passion palmarosa oil (e.g., Cymbopogon martini); patchouli (e.g., Pogostemon cablin); p-cymene; pennyroyal oil; pepper; peppermint (e.g., Mentha piperita); perillaldehyde; petitgrain (e.g., Citrus aurantium amara); phenyl ethyl alcohol; phenyl ethyl propionate; phenyl ethyl-2-methylbutyrate; pimento berry; pimento leaf; pinane hydroperoxide; pinanol; pine ester; pine needle; pine; pinene; piperonal; piperonyl acetate; piperonyl alcohol; plinol; plinyl acetate; pseudo ionone; rhodinol; rhodinyl acetate; rosalin; rose; rosemary (e.g., Rosmarinus officinalis); ryu; sage; sandalwood (e.g., Santalum album); sandenol; sassafras; sesame; soybean; spearmint; spice; spike lavender; spirantol; starflower; tangerine; tea seed; tea tree; terpenoid; terpineol; terpinolene; terpinyl acetate; tert-butylcyclohexyl acetate; tetrahydrolinalool; tetrahydrolinalyl acetate; tetrahydromyrcenol; thulasi; thyme; thymol; tomato; trans-2-hexenol; trans-anethole and metabolites thereof; turmeric; turpentine; vanillin (e.g., 4-hydroxy-3-methoxy benzaldehyde); vetiver; vitalizair; white cedar; white grapefruit; wintergreen (methyl salicylate) and the like.

Further examples of suitable essential oils or their constituents may include, but are not limited to, members selected from the group consisting of aldehyde C16 (pure), .alpha.-terpineol, amyl cinnamic aldehyde, amyl salicylate, anisic aldehyde, benzyl alcohol, benzyl acetate, cinnamaldehyde, cinnamic alcohol, carvacrol, carveol, citral, citronellal, citronellol, dimethyl salicylate, eucalyptol (cineole), eugenol, isoeugenol, galaxolide, geraniol, guaiacol, ionone, d-limonene, menthol, methyl anthranilate, methyl ionone, methyl salicylate, .alpha.-phellandrene, pennyroyal oil, perillaldehyde, 1- or 2 phenyl ethyl alcohol, 1- or 2-phenyl ethyl propionate, piperonal, piperonyl acetate, piperonyl alcohol, D-pulegone, terpinen-4-ol, terpinyl acetate, 4-tert butylcyclohexyl acetate, thyme oil (white and red), thymol, trans-anethole, vanillin, ethyl vanillin, and the like.

Sawdust—in the context of the present technology, sawdust includes particles of untreated softwood or hardwood lumber with an average moisture content of less than about 12%, preferably between about 6% and about 8%. The particles range in size from about 0.5 mm to about 2.0 mm in thickness and about 0.5 mm to about 1.0 cm long. The preferred sawdust is one or more of spruce (Picea), pine (Pinus), Douglas fir (Pseudotsuga), true fir (Abies) or hemlock (Tsuga). Selected hardwoods include beech (Fagus), maple (Acer), alder (Alnus), birch (Betula) and oak (Quercus).

Diatomaceous earth—in the context of the present technology, diatomaceous earth is a naturally occurring, soft, siliceous sedimentary rock. It has a particle size ranging from less than 3 μm to more than 1 mm, but typically 10 to 200 μm. The typical chemical composition of oven-dried diatomaceous earth is 80-90% silica, with 2-4% alumina (attributed mostly to clay minerals) and 0.5-2% iron oxide combined with other natural clays and minerals.

Household container—in the context of the present technology, a household container includes a green bin, compostable matter collection container, garbage can, trash can and any other container that is used to collect food scraps and other organic waste.

Compostable matter—in the context of the present technology, compostable matter is any organic matter that can be collected in a green bin. The most common compostable matter is food scraps. The following is a non-exhaustive list of compostable matter: fruit and vegetable scraps; food leftovers, plate scrapings; meat, fish, giblets and bones; dairy products, butter, mayonnaise; eggshells and seafood shells; bread, cereal, grains; pasta, pizza; baked goods, candies; soiled paper towels soiled tissues; paper towels, paper food packaging; newsprint; paper cups and paper plates; soiled paper food packaging; soiled newsprint; used paper cups and used paper plates; butcher and parchment papers; flour bags and sugar bags; coffee filters and coffee grounds; paper tea bags and tea leaves; solidified fats and solidified grease; baking ingredients, herbs, spices; houseplants, cut flowers and dried flowers; nuts, pits, seeds and shells; and wooden stir sticks and wooden chop sticks.

### DETAILED DESCRIPTION

A composition for reducing odors in green bins and other household containers includes odor reducing components and at least one odor masking component. In one embodiment, the composition is a dry composition consisting of about 25 to about 48 percent w/w sawdust, about 15 to about 25 percent w/w zeolite, and about 25 to about 28 percent w/w diatomaceous earth.

In one embodiment, the composition is a dry composition consisting of about 25 to about 40 percent w/w sawdust, about 15 to about 25 percent w/w zeolite, about 25 to about 28 percent w/w diatomaceous earth about 5 percent w/w sodium bicarbonate and about 2 percent w/w essential oil.

In one embodiment, the composition is a dry composition consisting of about 36 percent w/w Douglas fir sawdust, about 18 percent w/w zeolite, about 36 percent w/w diatomaceous earth, about 9 percent w/w sodium bicarbonate and about 1 percent w/w essential oil.

In one embodiment, the essential oil is organic lavender oil.

In one embodiment, the composition is a dry composition consisting of about 25 to about 40 percent w/w sawdust, about 15 to about 25 percent w/w zeolite, about 25 to about 40 percent w/w diatomaceous earth and about 1 percent w/w essential oil.

In one embodiment, the composition is a dry composition consisting of about 25 to about 40 grams sawdust, about 15 to about 25 grams zeolite, about 25 to about 40 grams diatomaceous earth, about 5 grams sodium bicarbonate and about 1 gram essential oil.

In one embodiment, the composition is a dry composition consisting of about 15 to about 25 percent w/w zeolite, about 25 to about 40 percent w/w diatomaceous earth, about 1 percent w/w essential oil and the remainder being sawdust.

In one embodiment, the composition is a dry composition consisting of about 25 to about 40 percent w/w sawdust, about 25 to about 40 percent w/w diatomaceous earth, about 1 percent w/w essential oil and the remainder being zeolite.

In one embodiment, the composition is a dry composition consisting of about 25 to about 40 percent w/w sawdust, about 15 to about 25 percent w/w zeolite, about 1 percent w/w essential oil and the remainder being diatomaceous earth.

In one embodiment, the composition is a dry composition consisting of about 15 to about 25 percent w/w zeolite, about 25 to about 40 percent w/w diatomaceous earth, about 5 to about 10 percent w/w sodium bicarbonate, about 1 percent w/w essential oil and the remainder being sawdust.

In one embodiment, the composition is a dry composition consisting of about 25 to about 40 percent w/w sawdust, about 25 to about 40 percent w/w diatomaceous earth, about 5 to about 10 percent w/w/w sodium bicarbonate, about 1 percent w/w essential oil and the remainder being zeolite.

In one embodiment, the composition is a dry composition consisting of about 25 to about 40 percent w/w sawdust, about 5 to about 10 percent w/w/w sodium bicarbonate, about 1 percent w/w essential oil and the remainder being diatomaceous earth.

In one embodiment, the composition is a dry composition consisting of about 36 grams Douglas fir sawdust, about 18 grams zeolite, about 36 grams diatomaceous earth, and about 1 gram essential oil. In another embodiment, the composition further comprises about 9 grams sodium bicarbonate.

In one embodiment, the composition is a dry composition consisting of about 25 to about 40 grams sawdust, about 15 to about 25 grams zeolite, about 25 to about 28 grams diatomaceous earth, about 5 to about 10 grams sodium bicarbonate and about 1 gram essential oil.

In one embodiment, the composition has an average moisture content of less than about 12%.

In one embodiment, the desired composition can be achieved by individually weighing out each component followed by transfer to an industrial mixer or blender, wherein they are subsequently blended. In another embodiment, the mixer or blender may be positioned on a weigh-scale so that each component can be added directly to the mixing device, thus eliminating a separate weigh step and transfer step, and thus providing a more reliable and accurate batching process. Examples of blenders and mixers that may be used to formulate the compositions of the present invention include, but are not limited to, ribbon blenders, V-blenders, cone screw blenders, screw blenders, double cone blenders, planetary mixers, dispersion mixers, counter-rotating mixers, paddle mixers, jet mixers, drum blenders, Banbury mixers, and combinations thereof.

Mixing may be achieved in a batch mixing process or in a continuous process. A single mixing device may be used, or two or more in series and/or in parallel may be used. The various solid components may be added using standard means known to one of ordinary skill in the art, for example single-screw or twin-screw extruders. The finished product has an unlimited shelf life when packaged in a water resistant or waterproof package.

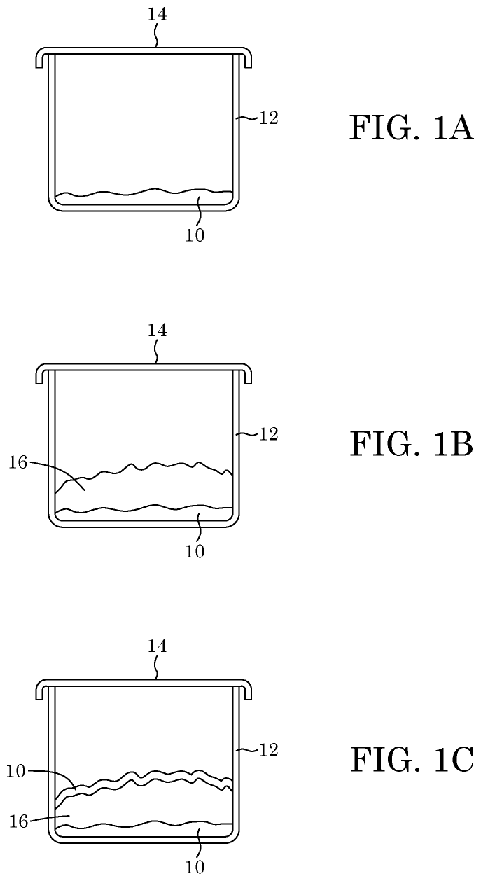

In one embodiment, as shown in FIG. 1A, the composition 10 is provided in a container 12 with a lid 14 or is added to a household container 12 with a lid 14. As shown in FIG. 1B, food waste/compostable matter 16 is then added to the container 12 and, as shown in FIG. 1C, more of the composition 10 is sprinkled on top of the food waste 16. FIG. 1C therefore represents the storage mode. The ratio of food scraps to composition is about 40 to about 1. About 75 grams to about 100 grams of the composition is added to the container 12.

The composition both controls and masks odor from compostable matter stored in household containers. When the compostable matter that has been stored in the household container is later composted, the composition functions as a compost amendment. Without being bound to theory, zeolite provides a source of slowly released potassium. Without being bound to theory, the addition of diatomaceous earth to soil amended with fertilizer may allow for more efficient use of fertilizer and reduce the impact that fertilizer has on the environment through leaching. Without being bound to theory, efficient and fast decomposition is based on the carbon to nitrogen (C:N) ratio of the material. A higher C:N ratio slows decomposition, while too low of a C:N results in NH3 (ammonia) release and rise in pH which kills of microorganisms. The ideal C:N ratio of a compost pile is 25-30:1. Fruit waste has a C:N ratio of about 35:1 and vegetable waste has a C:N ratio of about 12-25:1.

### Example 1

Three separate tests were conducted as follows:

A first empty, clean green bin had a layer of the composition shaken into it. A combination of: vegetable peelings, which included carrot peels and potato peels; vegetable scraps, including outer leaves of Brussel sprouts and broccoli stems; and apple cores was placed on top of the composition. A small amount of the composition was shaken on top of the combination of food scraps. The lid of the first green bin was closed. The first green bin was kept for 2 days at between 20° C. and 25° C.

A second empty, clean green bin did not have the composition in it, and only had the same combination of vegetable peelings, vegetable scraps and apple cores. The lid of the second green bin was closed. The second green bin was also kept for 2 days at between 20° C. and 25° C.

The lids of both green bins were opened and four people sniffed the green bins. It was reported that the first green bin with the composition produced less malodor. The lids were replaced and kept for another 2 days at between 20° C. and 25° C. The lids of both green bins were then opened and again four people sniffed the green bins. It was reported that the first green bin with the composition still produced less malodor, that the malodor had not increased significantly over the two days and the malodor from the second green bin was significantly higher than it had been two days previously. Both bins were then emptied.

### Example 2

Three separate tests were conducted as follows:

A first empty, clean green bin had a layer of the composition shaken into it. A combination of: vegetable peelings, including carrot peels and potato peels; vegetable scraps, including outer leaves of Brussel sprouts and broccoli stems; and apple cores was placed on top of the composition. A small amount of the composition was shaken on top of the combination. The lid of the green bin was kept off. The green bin was kept for 2 days at between 20° C. and 25° C.

A second empty, clean green bin did not have the composition in it, and only had the same combination of vegetable peelings, vegetable scraps and apple cores. The lid of the second green bin was kept off. The second green bin was also kept for 2 days at between 20° C. and 25° C.

After two days, the second green bin had a swarm of fruit flies while the first green bin had a small number of fruit flies.

### Example 3

Three separate tests were conducted as follows:

A first empty, clean green bin had a layer of the composition shaken into it. A combination of: vegetable peelings, including carrot peels and potato peels; vegetable scraps, including outer leaves of Brussel sprouts and broccoli stems; and apple cores was placed on top of the composition. A small amount of the composition was shaken on top of the combination of food scraps. The lid of the first green bin was closed. The first green bin was kept for 2 days at between 20° C. and 25° C. A combination of: vegetable peelings, including carrot peels and potato peels; vegetable scraps, including onions and peppers, and apple peel was placed on top of the composition, a small amount of the composition was shaken onto the food scraps and the lid was replaced.

A second empty, clean green bin did not have the composition in it, and only had the same combination of vegetable peelings, vegetable scraps and apple cores. The lid of the second green bin was closed. The second green bin was also kept for 2 days at between 20° C. and 25° C. after which time the combination of: vegetable peelings, including carrot peels and potato peels; vegetable scraps, including onions and peppers, and apple peel was placed on top of the vegetable scraps in the second green bin and the lid was replaced.

Both green bins were kept for another 2 days at between 20° C. and 25° C. after which time, more food scraps were added, the composition was shaken on top of the food scraps in the first green bin and both lids were replaced.

The lids of both green bins were opened and four people sniffed the green bins. It was reported that the first green bin with the composition produced less malodor. Both bins were then emptied.

### Example 4

Three separate tests were conducted as follows:

A first empty, clean green bin had a layer of the composition shaken into it. A mixture of at least five of the following where added on top of the composition: fruit and vegetable scraps; food leftovers, plate scrapings; meat, fish, giblets and bones; dairy products, butter, mayonnaise; eggshells and seafood shells; bread, cereal, grains; pasta, pizza; baked goods, candies; soiled paper towels soiled tissues; paper towels, paper food packaging; newsprint; paper cups and paper plates; soiled paper food packaging; soiled newsprint; used paper cups and used paper plates; butcher and parchment papers; flour bags and sugar bags; coffee filters and coffee grounds; paper tea bags and tea leaves; solidified fats and solidified grease; baking ingredients, herbs, spices; houseplants, cut flowers and dried flowers; nuts, pits, seeds and shells; wooden stir sticks; and wooden chop sticks, generally referred to as compostable matter. A small amount of the composition was shaken on top of the combination of the compostable matter. The lid of the first green bin was closed. The first green bin was kept for 2 days at between 20° C. and 25° C.

A second empty, clean green bin did not have the composition in it, and only had the same compostable matter as the first green bin. The lid of the second green bin was closed. The second green bin was also kept for 2 days at between 20° C. and 25° C.

The lids of both green bins were opened and four people sniffed the green bins. It was reported that the first green bin with the composition produced less malodor. The lids were replaced and kept for another 2 days at between 20° C. and 25° C. The lids of both green bins were then opened and again four people sniffed the green bins. It was reported that the first green bin with the composition still produced less malodor, that the malodor had not increased significantly over the two days and the malodor from the second green bin was significantly higher than it had been two days previously. Both bins were then emptied.

### Example 5

Three separate tests were conducted as follows:

A first empty, clean green bin had a layer of the composition shaken into it. A combination of: vegetable peelings, including carrot peels and potato peels; vegetable scraps, including outer leaves of Brussel sprouts and broccoli stems; and apple cores was placed on top of the composition. A small amount of the composition was shaken on top of the combination. The lid of the first green bin was closed. The first green bin was kept for 2 days at between 20° C. and 25° C. The first green bin was emptied into a first composter. The first green bin was rinsed, air dried and the process of adding the composition, adding food scraps and shaking more composition onto the food scraps was repeated five times with different food scraps as were available. The contents of the first green bin were emptied into the first composter each time.

A second empty, clean green bin did not have the composition in it, and only had the same combination of vegetable peelings, vegetable scraps and apple cores. The lid of the second green bin was closed. The second green bin was also kept for 2 days at between 20° C. and 25° C. The second green bin was emptied into a second composter. The second green bin was rinsed, air dried and the process of adding food scraps was repeated five times with different food scraps as were available. The contents of the second green bin were emptied into the second composter each time.

At the end of the collection period, the contents of the green bins were allowed to cure for 21 days. After that time it was found that the compost produced from the food scraps from the first green bin met one or more of the following requirements:

a) the respiration rate is less than, or equal to, 400 milligrams of oxygen per kilogram of volatile solids (or organic matter) per hour;
b) the carbon dioxide evolution rate is less than, or equal to, 4 milligrams of carbon in the form of carbon dioxide per gram of organic matter per day; and
c) the temperature rise of the compost above ambient temperature is less than 8° C., indicating that the compost was mature and stable.

In contrast, the compost produced from the food scraps from the second green bin did not meet the requirements.

### Example 6

Three separate tests were conducted as follows:

A first empty, clean household container had a layer of the composition shaken into it. A mixture of at least five of the following where added on top of the composition: fruit and vegetable scraps; food leftovers, plate scrapings; meat, fish, giblets and bones; dairy products, butter, mayonnaise; eggshells and seafood shells; bread, cereal, grains; pasta, pizza; baked goods, candies; soiled paper towels soiled tissues; paper towels, paper food packaging; newsprint; paper cups and paper plates; soiled paper food packaging; soiled newsprint; used paper cups and used paper plates; butcher and parchment papers; flour bags and sugar bags; coffee filters and coffee grounds; paper tea bags and tea leaves; solidified fats and solidified grease; baking ingredients, herbs, spices; houseplants, cut flowers and dried flowers; nuts, pits, seeds and shells; wooden stir sticks; and wooden chop sticks, generally referred to as compostable matter. A small amount of the composition was shaken on top of the compostable matter. The lid of the first household container was closed. The first household container was kept for 2 days at between 20° C. and 25° C. The first household container was emptied into a first composter. The first household container was rinsed, air dried and the process of adding the composition, adding compostable matter and shaking more composition onto the compostable matter was repeated five times with different compostable matter as was available. The contents of the first household container were emptied into the first composter each time.

A second empty, clean household container did not have the composition in it, and only had the same compostable matter as the first household container. The lid of the second household container was closed. The second household container was also kept for 2 days at between 20° C. and 25° C. The second household container was emptied into a second composter. The second household container was rinsed, air dried and the process of adding compostable matter was repeated five times with different compostable matter as was available. The contents of the second household container were emptied into the second composter each time.

At the end of the collection period, the contents of the household containers were allowed to cure for 21 days. After that time it was found that the compost produced from the compostable matter from the first household container met one or more of the following requirements:

a) the respiration rate is less than, or equal to, 400 milligrams of oxygen per kilogram of volatile solids (or organic matter) per hour;
b) the carbon dioxide evolution rate is less than, or equal to, 4 milligrams of carbon in the form of carbon dioxide per gram of organic matter per day; and
c) the temperature rise of the compost above ambient temperature is less than 8° C., indicating that the compost was mature and stable.

In contrast, the compost produced from the compostable matter from the second household container did not meet the requirements.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A dry composition for controlling food scrap odor, the dry composition comprising at least one sawdust, at least one zeolite and at least one diatomaceous earth.

2. The dry composition of claim 1 further comprising an essential oil.

3. The dry composition of claim 2, the composition having an average moisture content of less than about 12%.

4. The dry composition of claim 2, wherein the sawdust is a softwood sawdust.

5. The dry composition of claim 3, wherein the sawdust is one or more of Tsuga, Picea, Pinus, Abies and Pseudotsuga sawdust.

6. The dry composition of claim 4, wherein the sawdust is Pseudotsuga sawdust.

7. The dry composition of claim 6, wherein the essential oil is lavender oil.

8. The dry composition of claim 5 consisting of about 15 to about 25 percent w/w zeolite, about 25 to about 40 percent w/w diatomaceous earth, about 5 to about 10 percent w/w sodium bicarbonate, about 1 percent w/w essential oil and the remainder being the sawdust.

9. The dry composition of claim 5 consisting of about 25 to about 40 percent w/w sawdust, about 25 to about 40 percent w/w diatomaceous earth, about 5 to about 10 percent w/w sodium bicarbonate, about 1 percent w/w essential oil and the remainder being zeolite.

10. The dry composition of claim 5 consisting of about 25 to about 40 percent w/w sawdust, about 5 to about 10 percent w/w/w sodium bicarbonate, about 1 percent w/w essential oil and the remainder being diatomaceous earth.

11. The dry composition of claim 5 comprising about 36 percent w/w sawdust, about 18 percent w/w zeolite, about 36 percent w/w diatomaceous earth, about 9 percent w/w sodium bicarbonate and about 1 percent w/w essential oil.

12. A method of reducing and masking odor from compostable matter collected in a household container, the method comprising: adding a composition consisting of at least one sawdust, at least one zeolite, at least one diatomaceous earth and at least one essential oil to the household container; and adding compostable matter to the container.

13. The method of claim 12, wherein the ratio of compostable matter to composition is about 40 to about 1.

14. The method of claim 13, wherein the compostable matter is food scraps.

15. A method of reducing and masking odor from compostable matter in a household container, the method comprising: adding a first amount of a composition comprising about 36 percent w/w sawdust, about 18 percent w/w zeolite, about 36 percent w/w diatomaceous earth, about 9 percent w/w sodium bicarbonate and about 1 percent w/w essential oil to the household container; adding compostable matter to the household container; and adding a second amount of the composition on top of the compostable matter.

16. The method of claim 15, further comprising adding a second amount of compostable matter to the household container; and adding a third amount of the composition on top of the second amount of compostable matter.

17. The method of claim 16, further comprising sequentially adding compostable matter to the household container and sequentially adding the composition on top of the compostable matter.

18. The method of claim 17, wherein the ratio of compostable matter to composition is about 40 to about 1.

19. The method of claim 18, wherein the compostable matter is food scraps.
